(19) United States
(12) Patent Application Publication
(10) Pub. No.: US 2022/0117869 A1
(43) Pub. Date: Apr. 21, 2022

(54) TOPICAL SANITIZING COMPOSITIONS

(71) Applicant: Reckitt Benckiser Health Limited, Berkshire (GB)

(72) Inventors: Edward CRUDDEN, Montvale, NJ (US); Jennifer MUSYOKI, Montvale, NJ (US); Kelly Marie WHITEHEAD, Montvale, NJ (US)

(73) Assignee: Reckitt Benckiser Health Limited, Berkshire (GB)

(21) Appl. No.: 17/427,311

(22) PCT Filed: Feb. 11, 2020
(86) PCT No.: PCT/GB2020/050302

§ 371 Date: Jul. 30, 2021

Related U.S. Application Data
(60) Provisional application No. 62/803,654, filed on Feb. 11, 2019.

(51) Int. Cl. A61K 8/46; A61K 8/04; A61K 8/362; A61K 8/36; A61K 8/02; A61Q 17/00

(52) U.S. Cl. CPC A61K 8/463; A61K 8/046; A61K 8/362; A61K 2800/30; A61K 8/0208; A61Q 17/005; A61K 8/36

(57) ABSTRACT

Aqueous liquid topical compositions for topical application to a human or animal body, particularly to a human body and most particularly to the skin which compositions comprise a) 0.001% wt to 1% wt surfactant, b) at least one carboxylic acid or a salt thereof, and wherein the aqueous liquid compositions are at pH of about 4.7 or less and which provide effective sanitization against both gram positive and gram negative bacteria and is well tolerated by the skin, even after repeated use. Also provided are a dispenser and a disposable wipe containing a composition of the invention, the topical use of a composition of the invention to provide an antimicrobial benefit to skin and/or hair, and a method for providing an antimicrobial benefit to skin and/or hair by topically applying a composition of the invention.

The present invention relates to personal care products which are suitable for application to skin and/or hair, particularly to the skin and most particularly to the hands, to provide an antimicrobial (sanitizing) effect. More particularly, the compositions according to the invention are aqueous liquid compositions and especially aqueous liquid foaming compositions. The compositions may be applied to human or animal skin and/or hair but are especially intended for application to skin, especially human skin.

### BACKGROUND

Topical compositions, per se, are well-known in the cosmetic, dermatological as well as in the pharmaceutical fields. Topical compositions are intended to provide at least one specific benefit after being applied to the area which is desired to be treated by the user of the composition.

For example, personal care compositions which are primarily intended to provide an antimicrobial (sanitizing) effect to the area of skin to be treated after being topically applied to that area, and which do not require rinsing with water, are known in the field of personal care products.

Increasingly, consumers are aware of the health benefits of using such antimicrobial (sanitizing) products. Typically, such products are used to sanitize hands in many different situations. Such situations include: where there is not ready availability of soap and water (such as when travelling, or, in remote locations), where rapid sanitization is required (for example upon entering and leaving microbially sensitive areas and in certain work environments such as hospitals and nurseries) and where effective hand washing cannot be guaranteed (for example for young children, in schools and colleges). This is not an exhaustive list of situations where such sanitizing compositions are typically used as this will depend upon the situation in which the user finds him or herself.

The use of topically applied sanitizing compositions which can provide rapid and effective antimicrobial effects is therefore of increasing importance to consumers. For many people, topically applied sanitizing compositions (which can be used anywhere as they do not require the traditional method of using a soap bar or liquid soap to wash followed by rinsing with water and drying) provide a simple and effective way of maintaining hygiene. This in turn helps to reduce the spread of diseases caused by microbes and provides reassurance to the user that they are maintaining good standards of hygiene.

### PRIOR ART

Examples of sanitizing compositions which are intended for topical application, and which do not need to be rinsed with water, are disclosed in the literature.

Budhian et al in WO2017/072482 disclose treatment compositions which are to be used to impart an antimicrobial effect to animate and inanimate surfaces to be treated. The compositions comprise as an antimicrobial constituent at least one of lactic acid, citric acid, tartaric acid, the substituted acids thereof, derivatives thereof or salts thereof. Anionic surfactants are also used in the compositions.

Yuan et al in US 2008/0247960 disclose foaming topical compositions for application to the human body, particularly to the skin, which provide both a cleaning benefit and a durable antimicrobial benefit. The compositions comprise anionic surfactants with citric acid or lactic acid as an antimicrobial constituent.

De Szalay et al in WO2018/078336 disclose acidic female intimate cleansing compositions which have low irritation characteristics and provide good antimicrobial activity. These compositions contain lactic acid and an anionic constituent system which comprises secondary alkane sulfonate compound(s), an N-acyl sarcosinate compound and aromatic hydrotrope compound which boosts the antimicrobial activity of the lactic acid.

Rypkema et al in WO2006/027551 disclose a liquid composition for treatment of the skin and/or hair to provide a cleaning and/or sanitizing effect thereto. The compositions comprise sodium lauryl ether sulphate as surfactant, benzoic acid as a biocidal constituent with sodium lactate and citric acid included as buffers.

Bruning et disclose in WO2017/055789 feminine intimate personal wash compositions. The compositions comprise lactic acid to provide an antimicrobial affect but need to be rinsed off as they are personal wash composition. The compositions may comprise sodium laureth sulphate.

Tan et al disclose in WO2015/058942 compositions for liquid cleaning and disinfection which comprise the anionic surfactant SLES and two carboxylic acids, one of which is citric acid. The other is chosen from malonic acid, malic acid or glycolic acid. Comparative examples use citric acid in combination with lactic acid.

Cornford discloses in WO2013/185074 cleaning and sanitizing compositions which may optionally comprise an anionic surfactant. Other optional ingredients disclosed include an acid such as lactic acid or citric acid. However, no examples with these ingredients are provided.

Vermeulen et al in WO2013/101932 disclose antibacterial liquid cleansing compositions which comprise lactic acid/lactate and which are suitable for personal use. The compositions may also comprise anionic surfactant such as sodium laureth sulphate.

Liquid cleansing compositions comprising an antibacterial system comprising lactic acid or citric acid, and, at least 5% wt. surfactant are disclosed in WO2018/022016.

Skin or hair care compositions comprising a stabilizing acid, such as lactic acid, are disclosed in EP 1 593 371A1.

The use of organic acids for microbiological control is also known in applications outside of personal care applications.

For example, in WO01/64035, acidic antimicrobial compositions for treating food and food contact surfaces are disclosed. These compositions may comprise anionic surfactant and organic acids selected from citric acid, malic acid, benzoic acid and succinic acid.

WO01/94513 discloses biocidal cleaner compositions which comprise between 0.01 and 5% wt of an anionic surfactant chosen from specific types of anionic surfactant and an acid which may be selected from various carboxylic acids including citric acid and lactic acid.

In WO99/29815 dishwashing compositions comprising salts of carboxylic acids for antibacterial activity are disclosed.

Concentrated solutions of acid, which may comprise surfactant, are disclosed for use in the fermentation of hops in WO2015/136366 where concentrated citric acid solutions are exemplified, and, lactic acid is disclosed in the description.

### Problems the Invention Seeks to Address

The basic principle of personal washing/sanitization is recognized to encompass the need to control, or break, the potential chain of infection from person to person, or, from inanimate object to person. Handwashes which are used with water have been used for many years.

Whilst aqueous liquid compositions which do not require rinsing after use are also known in the art for sanitizing skin and/or hair, there remains a need for such compositions which provide a balance of several desirable characteristics.

As sanitizing compositions are often used in situations where frequent sanitization is required, the cumulative effect of this frequent use, even if mild at each use, can be significantly detrimental to the skin. With repeated use of such compositions the treated skin can become irritated and/or dry, sometimes severely. The frequent use of alcohol-based compositions on the skin may lead to disruption of the integrity of the skin mantel. This in turn may lead to infection.

Prior art compositions, especially those containing high levels of alcohol and/or harsh ingredients such as benzalkonium chloride, may tend to slightly irritate, and/or dry, the skin. These products may also have a noticeable ‘chemical’ odour leading to a perception by the user of the product being harsh, or drying, on the skin. This can deter some potential uses from using such compositions frequently or even at all.

Thus, there is a need in the art for sanitizing compositions which are well tolerated by the skin (even upon repeated use) and which do not cause unacceptable levels of irritation and/or dryness but which still provide beneficial levels of sanitation. There is also a need for sanitising compositions which users find convenient to use in a variety of situations. Most consumers find irritated and/or dry skin to be inconvenient and uncomfortable. This can deter the frequent use, or indeed any use, of such compositions. Thus, the provision of a composition which consumers can use to conveniently and effectively sanitize their skin and/or hair, even on a frequent basis, is desirable.

If sanitizing compositions are used on hair, the same considerations regarding dryness apply for the hair. Also, the considerations for the skin which is in contact with the hair being treated are as above.

Additionally, it is desirable to provide sanitizing compositions for personal use which do not require rinsing.

It is also desirable to provide effective levels of germ kill on both gram-positive bacteria (such as S. aureus) and gram-negative bacteria (such as E. coli). To provide effective sanitation, the compositions should ideally provide at least a 3 log₁₀ reduction of various microorganisms/bacteria when tested according to the standard test protocols of at least one of ASTM E2315-03 “Standard Guide for Assessment of Antimicrobial Activity Using a Time-Kill Procedure on gram-positive and/or gram-negative bacteria or EN 1276: 2009 method entitled “Chemical Disinfectants and Antiseptics—Quantitative Suspension Test for the Evaluation of bactericidal activity of chemical disinfectants and antiseptics used in food, industrial, domestic and institutional areas—Test method and requirements (Phase 2, step 1)”. Achieving this level of sanitation is typically difficult without including high levels of alcohol and/or biocidal ingredients in a personal care composition. Furthermore, it is desirable to provide these effective levels of germ kill in contact times which the user will find convenient, such as up to 60 seconds or preferably up to 30 seconds.

It is also desirable when applying and using sanitizing compositions for personal use, for the user to see, and ideally feel, the coverage of the composition on the area being treated to maximise sanitization efficacy. Therefore, there is also a need in the art for personal use sanitization products which can be monitored visually and/or through feel by the user to ensure adequate coverage of the area to be treated.

Furthermore, sanitizing compositions for personal use are typically produced on an industrial scale. This requires the production process used to be sufficiently reliable and straightforward to allow for large scale production without unacceptable production difficulties. It is therefore desirable to provide sanitizing compositions for personal use which can be easily produced on an industrial scale and with usual industrial scale manufacturing tolerances. For example, it is desirable to provide such compositions which can be produced over a pH range which is commensurate with usual industrial scale manufacture tolerances.

Furthermore, the production of personal care sanitising compositions containing large concentrations of alcohol is typically associated with strict production practices as alcohols are considered flammable ingredients. Therefore, suitable production methods need to be followed and the appropriate warnings are required on the composition's packaging. Such compositions may also present problems in distribution because of their flammable nature. The provision of an effective sanitising composition for personal care which does not contain high concentrations of flammable material, and so can be more easily produced and transported than its high-alcohol alternative offers advantages to the producer of these compositions.

The present invention seeks to address one or more of the aforementioned problems.

In particular, the present invention seeks to provide sanitizing compositions for personal use, which can be effectively used on skin and/or hair, which are well tolerated by the skin/hair even after repeated use and which do not cause unacceptable levels of skin irritation, such as damage to the skin mantel and/or drying. In particular, the present invention seeks to provide sanitising compositions for personal care which do not have a strong odour.

It is also an object of the invention to provide sanitizing compositions for personal use, which can be used for sanitisation without the concurrent use of water.

It is also an object of the present invention to provide sanitising compositions for personal use which are convenient to use and/or can be used in a variety of situations.

It is also an object of the invention to provide sanitizing compositions for personal use, which can especially be used on skin and/or hair and which are effective on gram-negative bacteria and/or gram-positive bacteria, preferably within a contact time of up to 60 seconds.

It is also an object of the invention to provide sanitizing compositions for personal use, which can especially be used on skin and/or hair and which are effective on both gram-negative bacteria and/or gram-positive bacteria and which provide at least a 3 log₁₀ reduction of various microorganisms/bacteria when tested according to the standard test protocols of at least one of ASTM E2315-03 or EN 1276: 2009.

Another object of the invention is to provide sanitizing compositions for personal use which exhibit a visual cue and/or tactile cue during use to the user to aid the user in assessing if full coverage of the area to be treated has been achieved as the compositions can be moved to an area which appears not to be treated.

It is also an object of the present invention to provide sanitising compositions suitable for topical application which do not contain high levels of alcohol.

It is a further object of the invention to provide sanitizing compositions for personal use which are easily manufactured and/or transported on an industrial scale. Furthermore, it is an object of the present invention to provide personal sanitizing compositions which do not require their packaging to carry ‘flammable’ warning information.

Surprisingly, the inventors have found that one or more of the above problems can be ameliorated by compositions according to the present invention.

In particular, the present inventors have found that effective aqueous liquid sanitization compositions for personal use on skin and/or hair, including those which do not require rinsing after use, are obtained when formulated as according to the present invention. These compositions are formulated to be within a given pH range and to comprise the ingredients of the invention. Moreover, the inventors have found that their compositions provide good microbial control and typically provide at least a 3 log₁₀ reduction of various microorganisms/bacteria when tested according to the standard test protocols of at least one of ASTM E2315-03 or EN 1276: 2009 and are effective on gram-positive bacteria and/or gram-negative bacteria, typically both. The inventors have also found their compositions to provide effective hand sanitization effects with treatment times of up to 60 seconds, and typically, up to 30 seconds.

The compositions of the invention are convenient to use. They can be carried by the user and applied in a wide variety of situations as they am not reliant on the availability of water when formulated as a non-rinse composition.

The present inventors have found that such compositions do not cause unacceptable skin irritation or damage to the skin mantel. It has also been found that the compositions do not unacceptably dry the skin the skin even after repeated use. The compositions can be formulated so as not to comprise a high level of alcohol and therefore not exhibit a strong alcoholic odour.

The present inventors have also found that such compositions, which demonstrate foaming characteristics, are especially advantageous for achieving high coverage levels for the area(s) to be sanitized and thus aid the level of sanitation achieved. The foaming compositions of the invention also exhibit a beneficial visual and/or tactile cue during use to help the user assess if full coverage of the area to be treated has been achieved.

The compositions can be easily manufactured and transported industrially. Also, the compositions can be formulated such that the packaging does not require flammable or other hazard warnings to be included.

### STATEMENT OF INVENTION

Accordingly, in first aspect of the invention there are provided aqueous liquid compositions which provide a topical antimicrobial benefit, which compositions comprise:

a) 0.001% wt. to 1% wt. surfactant,
b) at least one carboxylic acid or a salt thereof, and
wherein the aqueous liquid compositions are at pH of about 4.7 or less.

It is preferred that the compositions of the inventions are foaming compositions.

It is further preferred that the compositions of the invention comprise water in an amount in the range of from 85% wt to 99% wt, based on the total weight of the composition.

It is also preferred that the compositions of the invention comprise 0.05% wt to 0.98% wt. surfactant based on the total weight of the composition.

In one embodiment of the invention, it is preferred that the compositions comprise anionic surfactant, and preferably the surfactant constituent in the compositions consists essentially of anionic surfactant.

Preferably the anionic surfactant comprises alkyl sulfates or salts thereof, especially alkyl ether sulfates, or salts thereof, most preferably selected from the group consisting of C₈-C₁₈ alkyl sulfates and their salts, and C₈-C₁₈ alkyl ether sulfates and their salts, such as lauryl sulfate and its salts and lauryl ether sulphate and its salts. Most preferred surfactants according to the invention are sodium lauryl sulfate and sodium lauryl ether sulphate.

In a further embodiment of the invention, it is preferred that the compositions comprise nonionic surfactant and/or cationic surfactant and/or amphoteric surfactant.

Preferably the compositions comprise less than about 5% wt of the carboxylic acid or salt thereof. It is preferred that the compositions comprise an amount of from 0.1% wt. to 4.5% wt of the carboxylic acid or salt thereof.

It is especially preferred that the compositions comprise a monocarboxylic acid or salt thereof. It is also especially preferred that the compositions comprise a tricarboxylic acid or salt thereof.

It is preferred that the compositions comprise a monocarboxylic acid or salt thereof in an amount in the range of from 0.1% wt. to 4.5% wt. It is also preferred that the compositions comprise a tricarboxylic acid or salt thereof in an amount in the range of from 0.1% wt. to 4.5% wt. According to one embodiment of the invention one or more monocarboxylic acids or salt thereof and one or more tricarboxylic acids or salt thereof may be present in the compositions. An especially preferred combination of carboxylic acids or salt thereof according to the present invention is the combination of citric acid or salt thereof and lactic acid or salt thereof as the carboxylic acid constituent.

The inventors have also found that especially good results are obtained when the compositions comprise a tricarboxylic acid or salt thereof and a monocarboxylic acid or salt thereof in at least a 1:1 weight, more preferably in the weight ratio of from 10:1 to 1:1, most preferably in a weight ratio of from 6:1 to 1.5:1, such as 5:1 to 2:1, for example 4.5:1 to 2.5:1, especially 4:1 to 3:1.

It is preferred that the compositions of the invention are at a pH of about 4.6 or less.

It is preferred that when the compositions of the invention comprise at least one monocarboxylic acid and at least one tricarboxylic acid, the compositions have a pH in the range of from 3 to about 4.6, more preferably from about 3.5 to about 4.5.

According to a second aspect of the invention there is provided a dispenser containing a composition according to the invention.

According to a third aspect of the invention, there is provided a disposable wipe comprising a composition of the invention.

According to a fourth aspect of the invention there is provided the use of a composition according to the invention to provide an antimicrobial benefit to sanitize skin and/or hair by the topical application of the composition to the skin and/or hair.

According to a fifth aspect of the invention there is provided a method for providing an antimicrobial benefit to skin and/or hair desired to be sanitized, comprising the steps of topically applying a composition according to the invention to the area of skin and/or hair to be sanitized in a suitable amount to provide a sanitizing effect, and, allowing contact between the composition and the skin and/or hair for sufficient time to provide the antimicrobial effect thereon.

Other features and advantages of the present invention will be apparent from the following detailed description of the invention and from the accompanying claims.

### DETAILED DESCRIPTION OF THE INVENTION

The compositions according to the present invention will now be described in more detail.

The compositions of the invention are aqueous liquid compositions suitable for application to the skin and/or hair, especially the skin. Preferably the compositions are aqueous liquid foaming compositions.

The term “foaming” as used herein means the compositions foam during use, even if they do not appear as a foam prior to use, for example when held in a dispenser. Foam present during use is often perceived by the user as a visual and/or tactile cue of the efficacy/activity of a composition. It also provides the advantage for the user that the coverage of the composition over the area to be treated can be visually or tactically assessed which aids the effectiveness of sanitization and reduces the likelihood of areas not being treated.

The term “antimicrobial” as used herein means that the control of at least one of bacteria, fungi and/or virus growth. “antibacterial” as used herein means the control of bacterial growth.

The amounts of ingredients stated herein refers to the amount of active ingredient based on the total weight of the composition. Unless otherwise stated, all weights are as wt % based on the total weight of the composition.

### Water

The liquid compositions of the invention are aqueous in nature. Water is included in the compositions to provide the balance of the composition to 100% by weight of the composition and typically forms the major component of it. The compositions typically comprise water in an amount in the range of from 85% wt to 99% wt, based on the total weight of the composition. Preferably the compositions comprise an amount of from 90% wt to 98.5% wt water, more preferably of from 93% wt to 98% wt, even more preferably in the range of from 94% wt to 97.5 wt %, such as 95% wt to 97% wt.

The water may be tap water but is preferably distilled water and is most preferably deionized water or “soft” water. If the water is tap water, it is preferably substantially free of any undesirable impurities such as organics or inorganics, especially minerals salts which are present in hard water which may thus undesirably interfere with the operation of the constituents present in the topical compositions according to the present invention.

### Surfactant

The compositions of the invention comprise include one or more surfactants, especially one or more anionic surfactants (and/or salt forms thereof). According to one aspect of the present invention it is preferred that the surfactant constituent consists essentially of anionic surfactant. According to one especially preferred embodiment of the present invention, the compositions comprise only anionic surfactants as the surfactant constituent. In a further embodiment of the invention, the compositions may contain nonionic surfactant and/or amphoteric (zwitterionic) surfactant and/or cationic surfactant. This may be in addition to the anionic surfactant (except for the cationic surfactant which if used is used as an alternative to the anionic surfactant). Suitable combinations of surfactant also include only nonionic surfactant and cationic surfactant, or, only nonionic surfactant and anionic surfactant as the surfactant constituent. The amphoteric surfactant, if used, may be used with any other surfactant type.

a) Anionic Surfactant

Preferably the compositions comprise one or more anionic surfactants which provide good foaming characteristics in use, especially anionic sulfate surfactants. By way of non-limiting example especially preferred anionic surfactants which provide such functions are alkyl sulfates, especially alkyl ether sulfates. One group of preferred anionic surfactants are C₈-C₁₈ alkyl sulfates, especially C₁₀-C₁₆ alkyl sulfates such as C₁₂-C₁₄ alkyl sulfates and their salts. Any suitable salt may be used and typically an alkali metal (such as sodium, potassium or lithium) or alkaline earth metal salt is used. Most preferably the sodium salt is used. A preferred anionic surfactant is sodium lauryl sulfate known as SLS. Especially preferred anionic surfactants are C₈-C₁₈ alkyl ether sulfates, especially C₁₀-C₁₆ alkyl ether sulfates such as C₁₂-C₁₄ alkyl ether sulfates and their salts. Again, any suitable salt may be used and typically an alkali metal (such as sodium, potassium or lithium) or alkaline earth metal salt is used. Most preferably the sodium salt is used. An especially preferred anionic surfactant is sodium lauryl ether sulphate known as SLES. Such foaming anionic surfactants, particularly the preferred alkyl ether sulfates, provide good foaming characteristics to the compositions and acceptable skin tolerance characteristics.

As such it is highly preferred that the compositions of the invention comprise anionic surfactant and especially one or more anionic surfactants based on alkyl sulfates, particularly alkyl ether sulfates and especially one or more of those as described in the following Examples. Desirably a major proportion of the anionic surfactant constituent consists of one or more alkyl sulfates especially alkyl ether sulfates, preferably at least 80% wt., more preferably at least 90% wt, and yet more preferably at least 95% wt. of the anionic surfactant constituent comprises alkyl sulfates especially alkyl ether sulfates. It is especially preferred that the anionic surfactant constituent comprises at least 99% wt., of one or more alkyl sulfates especially one or more alkyl ether sulfates. Most preferably the surfactant constituent consists essentially of one or more alkyl sulfates especially one or more alkyl ether sulfates. Combinations of one or more alkyl sulfates with one or more alkyl ether sulfates may also be used.

Examples of other anionic surfactants which may be used according to the present invention include alcohol sulfates and sulfonates, alcohol phosphates and phosphonates, alkyl ester sulfates, alkyl diphenyl ether sulfonates, sulfate esters of an alkyl phenoxy polyoxyethylene ethanol, alkyl monoglyceride sulfates, alkyl sulfonates, alpha-olefin sulfonates, beta-alkoxy alkane sulfonates, alkyl ether sulfonates, ethoxylated alkyl sulfonates, alkylaryl sulfonates, alkylaryl sulfates, alkyl monoglyceride sulfonates, alkyl carboxylates, alkyl ether carboxylates, alkyl alkoxy carboxylates having 1 to 5 moles of ethylene oxide, alkyl polyglycol ether sulfates (containing up to 10 moles of ethylene oxide), sulfosuccinates, octoxynol or nonoxynol phosphates, taurates, fatty taurides, fatty acid amide polyoxyethylene sulfates, acyl glycerol sulfonates, fatty oleyl glycerol sulfates, alkyl phenol ethylene oxide ether sulfates, paraffin sulfonates, alkyl phosphates, isethionates, N-acyl taurates, alkyl succinamates and sulfosuccinates, alkyl polysaccharide sulfates, alkyl polyglucoside sulfates, alkyl polyethoxy carboxylates, and sarcosinates or mixtures thereof.

Further examples of anionic surfactants include water soluble salts or acids of the formula (ROSO₃)ₓM or (RSO₃)ₓM wherein R is preferably a C₆-C₂₄ hydrocarbyl, preferably an alkyl or hydroxyalkyl having a C₁₀-C₂₀ alkyl component, more preferably a C₁₂-C₁₈ alkyl or hydroxyalkyl, and M is H or a mono-, di- or tri-valent cation, e. g., an alkali metal cation (e. g., sodium, potassium, lithium), or ammonium or substituted ammonium (e. g., methyl-, dimethyl-, and trimethyl ammonium cations and quaternary ammonium cations, such as tetramethyl-ammonium and dimethyl piperidinium cations and quaternary ammonium cations derived from alkylamines such as ethylamine, diethylamine, triethylamine, and mixtures thereof, and the like) and x is an integer, preferably 1 to 3, most preferably 1. Materials sold under the Hostapur and Biosoft trademarks are examples of such anionic surfactants.

Still further examples of anionic surfactants which may find use in the compositions include alkyl-diphenyl-ether sulphonates and alkyl-carboxylates. Other anionic surfactants can include salts (including, for example, sodium, potassium, ammonium, and substituted ammonium salts such as mono-, di- and triethanolamine salts) of soap, C₆-C₂₀ linear alkylbenzene sulfonates, C₆-C₂₂ primary or secondary alkanesulfonates, C₆-C₂₄ olefin sulfonates, sulfonated polycarboxylic acids prepared by sulfonation of the pyrolyzed product of alkaline earth metal citrates, C₆-C₂₄ alkyl polyglycol ether sulfates, alkyl ester sulfates such as C₁₄₋₁₆ methyl ester sulfates; acyl glycerol sulfonates, fatty oleyl glycerol sulfates, alkyl phenol ethylene oxide ether sulfates, paraffin sulfonates, alkyl phosphates, isethionates such as the acyl isethionates, N-acyl taurates, alkyl succinamates and sulfosuccinates, monoesters of sulfosuccinate (especially saturated and unsaturated C₁₂-C₁₈ monoesters) diesters of sulfosuccinate (especially saturated and unsaturated C₆-C₁₄ diesters), acyl sarcosinates, sulfates of alkyl polysaccharides such as the sulfates of alkyl polyglucoside, branched primary alkyl sulfates, alkyl polyethoxy carboxylates such as those of the formula RO(CH₂CH₂O)ₖCH₂COO⁻M⁺ wherein R is a C₈-C₂₂ alkyl, k is an integer from 0 to 10, and M is a soluble salt-forming cation.

Anionic compounds which function both as surfactants and as a hydrotrope may be included as either as part of the anionic surfactant constituent or as a co-surfactant. Exemplary hydrotropes include, inter alia, benzene sulfonates, naphthalene sulfonates, C₁-C₁₁ alkyl benzene sulfonates, naphthalene sulfonates, C₅-C₁₁ alkyl sulfonates, C₆-C₁₁ alkyl sulfates, alkyl diphenyloxide disulfonates, and phosphate ester hydrotropes. The hydrotropic compounds of the invention are often provided in a salt form with a suitable counterion, such as one or more alkali, or alkali earth metals, such as sodium or potassium, especially sodium. However, other water-soluble cations such as ammonium, mono-, di- and tri-lower alkyl, i.e., C₁₋₄ alkanol ammonium groups can be used in the place of the alkali metal cations. Exemplary alkyl benzene sulfonates include, for example, isopropylbenzene sulfonates, xylene sulfonates, toluene sulfonates, cumene sulfonates, as well as mixtures thereof. Exemplary C₅-C₁₁ alkyl sulfonates include hexyl sulfonates, octyl sulfonates, and hexyl/octyl sulfonates, and mixtures thereof. Particularly useful hydrotrope compounds include benzene sulfonates, o-toluene sulfonates, m-toluene sulfonates, and p-toluene sulfonates; 2,3-xylene sulfonates, 2,4-xylene sulfonates, and 4,6-xylene sulfonates; cumene sulfonates, wherein such exemplary hydrotropes are generally in a salt form thereof, including sodium and potassium salt forms.

Combinations of two or more anionic surfactants may also be used in the compositions of the invention if desired. However, according to one aspect of the present invention, it has been found beneficial for the compositions to comprise either an alkyl sulphate or an alkyl ether sulphate as the only anionic surfactant. In particular, sodium lauryl sulfate or sodium lauryl ether sulphate are especially preferred as the only anionic surfactant in the compositions, most especially sodium lauryl ether sulphate. Especially preferred according to the present invention is the use of an alkyl sulfate and salts thereof and/or an alkyl ether sulphate and salts thereof in the amounts stated herein. Most preferred is the use of lauryl sulfate and/or lauryl ether sulfate, especially the sodium salts thereof in the amounts stated herein.

b) Nonionic Surfactants

Exemplary useful nonionic surfactants are those which include a hydrophobic base portion, such as a long chain alkyl group or an alkylated aryl group, and a hydrophilic chain portion comprising a sufficient number of ethoxy and/or propoxy moieties to render the nonionic surfactant at least partially soluble or dispersible in water. By way of non-limiting example, such nonionic surfactants include ethoxylated alkylphenols, ethoxylated and propoxylated fatty alcohols, polyethylene glycol ethers of methyl glucose, polyethylene glycol ethers of sorbitol, ethylene oxide, propylene oxide block copolymers, ethoxylated esters of fatty (C₆-C₂₄) acids, condensation products of ethylene oxide with long chain amines or amides, and mixtures thereof. Further exemplary nonionic surfactants include, but are not limited to: methyl gluceth-10, PEG-methyl glucose distearate, PEG-20 methyl glucose sesquistearate, C₁₁-C₁₈ pareth-20, ceteth-8, ceteth-12, dodoxynol-12, laureth-15, PEG-20 castor oil, polysorbate 20, steareth-20, polyoxyethylene-10 cetyl ether, polyoxyethylene-10 stearyl ether, polyoxyethylene-20 cetyl ether, polyoxyethylene-10 oleyl ether, polyoxyethylene-20 oleyl ether, an ethoxylated nonylphenol, ethoxylated octylphenol, ethoxylated dodecylphenol, or ethoxylated fatty (C₆-C₂₂) alcohol, including 3 to 20 ethylene oxide moieties, polyoxyethylene-20 isohexadecyl ether, polyoxyethylene-23 glycerol laurate, polyoxyethylene-20 glyceryl stearate, PPG-10 methyl glucose ether, PPG-20 methyl glucose ether, polyoxyethylene-20 sorbitan monoesters, polyoxyethylene 80 castor oil, polyoxyethylene-15 tridecyl ether, polyoxyethylene-6 tridecyl ether, laureth-2, laureth-3, laureth-4, PEG-3 castor oil, PEG 600 dioleate, PEG 400 dioleate, and mixtures thereof. Other nonionic surfactants, although not specifically disclosed herein but known to the art may also be used. The nonionic surfactants may be present as single compounds or as mixtures of two or more nonionic surfactant compounds.

c) Amphoteric Surfactants

Exemplary useful amphoteric surfactants include derivatives of secondary and tertiary amines having aliphatic radicals that are straight chain or branched, and wherein one of the aliphatic substituents contains from about 8 to 18 carbon atoms and at least one of the aliphatic substituents contains an anionic water-solubilizing group, e.g., a carboxy, sulfonate, or a sulfate group. Non-limiting examples of compounds falling within this description include: sodium 3-(dodecylamino)propionate, sodium 3-(dodecylamino)propane-1-sulfonate, sodium 2-(dodecylamino)ethyl sulfate, sodium 2-(dimethylamino)octadecanoate, disodium 3-(N carboxymethyldodecylamino)propane-1-sulfonate, disodium octadecyliminodiacetate, sodium 1-carboxymethyl-2-undecylimidazole, and sodium N,N-bis(2-hydroxyethyl)-2-sulfato-3-dodecoxypropylamine. Further exemplary useful amphoteric surfactants include sarcosinates and taurates, amide sulfosuccinates, and betaines including phosphobetaines.

Exemplary useful betaine surfactants which may be represented by the general formula:

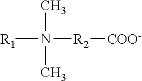

wherein: R₁ is an alkyl group containing from 8 to 18 carbon atoms, or the amido radical which may be represented by the following general formula:

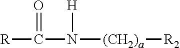

wherein: R is an alkyl group having from 8 to 18 carbon atoms, a is an integer having a value of from 1 to 4 inclusive, and R2 is a C₁-C₄ alkylene group.

Examples of preferred betaines are dodecyl dimethyl betaine, cetyl dimethyl betaine, dodecyl amidopropyldimethyl betaine, tetradecyldimethyl betaine, tetradecylamidopropyldimethyl betaine, dodecyldimethylammonium hexanoate and particularly cocoamidopropyl betaine.

If present, the betaine may be present in any effective amount, and are preferably present in amounts of from 0.01% wt. to 10% wt., preferably 0.1-8% wt., preferably from 0.5-5% wt. based on the total weight of the composition.

d) Cationic Surfactants

In certain embodiments of the invention cationic surfactants may be included in the compositions.

However, cationic surfactants are omitted from the composition if it comprises anionic surfactants as these two types of surfactant are incompatible because of the undesirable formation of complexes.

Cationic surfactants based on quaternary ammonium compounds independently provide antimicrobial effects and so can contribute to the overall germ-kill effect. According to one embodiment of the invention cationic quaternary ammonium surfactants are preferred such as alkylbenzyl dimethyl ammonium chlorides and dialkyl dimethyl ammonium chlorides.

The compositions of the invention comprise surfactant in a total amount of from about 0.001% wt. to about 1% wt. surfactant, preferably in the range of from 0.01% wt. to less than 1% wt., preferably 0.05% wt to 0.98% wt., more preferably 0.07% wt to 0.5% wt and especially 0.1% wt to 0.3% wt., such as 0.12% wt. to 0.25% wt based on the total weight of the composition of which they form a part. Preferably, the compositions of the invention comprise anionic surfactant in an amount within the above ranges.

### Carboxylic Acid

The compositions of the present invention comprise at least one carboxylic acid or a salt thereof. Preferably the compositions comprise at least two or more carboxylic acids or salt thereof. In some embodiments of the invention the acids are preferred to the salts. Combinations of acids and salts may also be used if desired. The carboxylic acids or salts thereof exhibit antimicrobial properties yet are suitable for use in personal care topical formulations.

For the sake of brevity, the references herein to any of the carboxylic acids (generic or specific) includes reference to any salts thereof.

The compositions of the present invention may comprise monocarboxylic acids, dicarboxylic acids and/or tricarboxylic acids and mixtures thereof. The salts of these acids may be used but in some embodiments of the invention preferably the acids are used.

Any skin and hair compatible monocarboxylic acids may be used including acetic acid, oxalic acid, lactic acid, malonic acid, tartronic acid, salicylic acid, butanoic acid and nicotinic acid. However, lactic acid is especially preferred according to the invention.

Any skin and hair compatible dicarboxylic acids may be used including succinic acid, tartaric acid, glutaric acid and adipic acid.

Any skin and hair compatible tricarboxylic acids may be used including citric acid and aconitic acid, however, citric acid is especially preferred according to the invention.

Mixtures of different groups of carboxylic acids may be used according to the present invention. An especially preferred mixture according to the present invention is a mixture of a monocarboxylic acid with a tricarboxylic acid. However, dicarboxylic acids could also be used with either monocarboxylic acids or tricarboxylic acids (including the salts of any of these acids). An especially preferred mixture of carboxylic acids or their salts is a mixture of lactic acid used with citric acid.

It is also possible to use a single carboxylic acid or salts thereof in the compositions of the invention. In such compositions it is preferred to use citric acid, lactic acid or succinic acid alone, especially either citric acid or lactic acid and most especially citric acid.

Generally, the total amount of the one or carboxylic acids present in the compositions is not in excess of about 5% wt. based on the total weight of the composition. It is preferred that the compositions comprise an amount of from 0.1% wt. to 4.5% wt of the carboxylic acid or salt thereof. Typically, the compositions of the invention comprise the one or carboxylic acids in a total amount in the range of from 0.5% wt. to 4% wt., more preferably 1% wt. to 3.5% wt., most preferably 1.25% wt to 3% wt., such as 1.5% wt to 2.75% wt. Particularly preferred carboxylic acid antimicrobial constituents according to the invention, and the preferred amounts thereof, are disclosed in one or more of the Examples.

According to one aspect of the present invention, the compositions contain a monocarboxylic acid, dicarboxylic acid or tricarboxylic acid as the only carboxylic acid constituent, that carboxylic acid being present in the amounts stated above. Preferably the compositions contain only a tricarboxylic acid as the only carboxylic acid constituent in the amounts stated above.

If the compositions comprise a mixture of carboxylic acids, the individual types may be present in any amount within the amounts above. If a mixture is used, it is especially preferred that the compositions comprise a mixture of a monocarboxylic acid and a tricarboxylic acid within the total amounts given above.

When a monocarboxylic acid and a tricarboxylic acid are both present, the compositions typically comprise the monocarboxylic acid in an amount in the range of from 0.1% wt. to 4.5% wt., more preferably 0.2% wt. to 4% wt., most preferably 0.5% wt to 3% wt., such as 0.75% wt to 2.5% wt, for example 0.5% wt to 1.5% wt and the tricarboxylic acid in an amount in the range of from 0.1% wt. to 4.5% wt., more preferably 0.2% wt. to 4.2% wt., most preferably 0.5% wt to 4 wt., such as 1% wt to 3.5% wt with the total amount being up to 5% wt of the total weight of the composition.

The inventors have observed that especially good results are obtained when the compositions of the invention comprise a tricarboxylic acid and a monocarboxylic acid in a 1:1 weight ratio, or, where the composition comprises more of the of tricarboxylic acid than of the monocarboxylic acid. It is especially preferred that the weight ratio of the tricarboxylic acid to the monocarboxylic acid is the weight ratio of from 10:1 to 1:1, more preferably of from 6:1 to 1.5:1, even more preferably of from 5:1 to 2:1, such as of from 4.5:1 to 2.5:1 and especially of from 4:1 to 3:1.

Preferably the citric acid and lactic acid are present in the aforementioned ratios. Especially preferred compositions comprise citric acid and lactic acid in equal amounts, but even more preferably more citric acid is present than lactic acid.

### pH of the Compositions

The inventors have also found that excellent antimicrobial efficacy is provided when the compositions of the invention are formulated to be within a specific acidic pH range of about 4.7 or less, more preferably in the range of from 4.6 or less, even more preferably in the range of from 3 to about 4.7, most preferably of from about 3 to about 4.6, such as about 3 to about 4.5, e.g. about 3 to 4.4 or 4.3. The lower limit for the pH range is typically about pH 3. However, the compositions of the invention are preferably formulated to be above a pH value of 3.2, preferably above pH 3.3 or 3.4. According to one aspect of the present invention, when the composition comprises both at least one monocarboxylic acid and at least one tricarboxylic acid the composition preferably has a pH in the range of from about 3 to about 4.3, more preferably in the range of from about 3.3 to about 4.3, most preferably from about 3.5 to about 4.3, especially from about 3.8 to about 4.25, such as from about 3.9 to about 4.2. It is necessary to balance antimicrobial activity and skin tolerance. Hence very low pH's (especially below pH 3) are avoided according to the invention as they are more likely to adversely affect the skin/hair potentially leading to skin irritation and/or damage.

When it is necessary or desirable to adjust the pH of the compositions of the invention (e.g. to provide a pH which is better tolerated by skin/air than the starting pH of the composition after preparation but prior to pH adjustment), one or more pH adjusting agents and/or one or more pH buffers may be included in the compositions in effective amounts to provide the desired pH. Immediately upon preparation the compositions of the invention are typically acidic and generally have a pH in the range of from 2 to 5, usually 2.5 to 4.5, such as 3 to 4.5 prior to any adjustment of the pH with a pH adjustment agent.

The pH of the compositions of the invention are those as measured at 20° C.

By way of non-limiting example suitable pH adjusting agents include phosphorus containing compounds, monovalent and polyvalent salts such as of silicates, carbonates, and borates, certain acids and bases, tartrates and certain acetates. The use of a base as a pH adjuster, such as a hydroxide, for example an alkali metal or alkaline earth metal hydroxide, is preferred according to the present invention. The use of sodium hydroxide is especially preferred according to the present invention as a pH adjustment agent and will typically be present in the compositions of the invention.

By way of further non-limiting example pH buffering compounds may also be used in the compositions and examples include the alkali metal phosphates, polyphosphates, pyrophosphates, triphosphates, tetraphosphates, silicates, metasilicates, polysilicates, carbonates, hydroxides, and mixtures of the same. Certain salts, such as the alkaline earth phosphates, carbonates, hydroxides, can also function as buffers. It may also be suitable to use as buffers such materials as aluminosilicates (zeolites), borates, aluminates and certain organic materials such as gluconates, succinates, maleates, and their alkali metal salts.

When present, the pH adjusting agent is present in an amount effective to adjust or maintain the pH of the inventive composition within a target pH range. Typically, the pH adjusting agent may be included in relatively minor amounts such as from 0.01-5% wt. dependent on the degree of pH adjustment required to achieve the desired pH. If included, they are desirably present in amounts from 0.1-4% wt, such as 1-3% wt. Exemplary, and preferred pH adjusting agents are described with reference to one or more of the following Examples.

### Optional Ingredients

The foaming or non-foaming topical compositions of the invention may include one or more further optional constituents which may be used to impart one or more desired esthetic or technical benefits to the topical compositions. Such optional constituents include additives and adjuvants which are conventional in the cosmetic, pharmaceutical or dermatological field, such as moisturisers, skin conditioning agents, fragrances, essential oils, colorants, preservatives, further antimicrobially active compounds or materials, foam boosters, humectants, opacifiers, antioxidants, chelating agents, thickener and light stabilizers including UV absorbers. The amounts of these various additives and adjuvants are those conventionally used in the field, and, for example, range from 0.01% to 10% of the total weight of the composition.

The topical compositions may include a fragrance constituent, which may be based on natural and synthetic fragrances and most commonly are mixtures or blends of a plurality of such fragrances, optionally in conjunction with a carrier such as an organic solvent or a mixture of organic solvents in which the fragrances are dissolved, suspended or dispersed. By way of non-limiting example, natural fragrances include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamon, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax) as well as other further extracts such as eugenol and menthol. Menthol may be advantageously included in that it also provides a cooling sensation when topically applied. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Lilial is less preferred than the others and according to one embodiment of the invention compositions which are free of lilial are preferred. Examples of suitable ketones are the ionones, alpha-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, labolanum oil and lavendin oil. A further useful material which finds use in the fragrance constituent is farnesol which is the common chemical name for 3,7,11-trimethyldodeca-2,6,10-trienol, which is commercially available from several sources and has found use in cosmetic compositions, primarily as a fragrance constituent. While not wishing to be bound by the following, it is suspected that the inclusion of farnesol may improve the antimicrobial efficacy of the compositions when they are topically applied and used in their normal manner.

When present in a composition, in accordance with certain of the preferred embodiments, the fragrance constituent may be present in any effective amount such that it can be discerned by a consumer of the topical composition. However, it is advantageously present in amounts of up to about 0.5% wt., preferably are present in amounts of from about 0.00001% wt. to about 0.3% wt., and most preferably is present in an amount of from about 0.0001% wt. to 0.25% wt. based on the total weight of the composition of which it forms a part.

The aqueous foaming or non-foaming topical compositions of the invention may include one or more constituents, particularly one or more essential oils, which are selected to provide a so-called “aromatherapy benefit” to the user. Essential oils am complex mixtures of different organic molecules, such as terpenes, alcohols, esters, aldehydes, ketones and phenols. Such essential oils are frequently extracted from naturally occurring botanical sources such as flowers, stems, leaves, roots and barks of aromatic plants. While essential oils may be used singly, it is also common to utilize blends of essential oils to provide a conjunctive aroma benefit, and possibly a therapeutic benefit as well.

A variety of essential oils providing an aromatherapy benefit may be incorporated into the topical compositions of the invention either as a single essential oil or as a mixture of two or more essential oils. It is also to be recognized when used, an essential oil providing an aromatherapy benefit may replace all or part of any further fragrance constituent including the fragrance constituents discussed above as many of the essential oils providing an aromatherapy benefit are pungent and odiferous. Such essential oils providing an aromatherapy benefit may be used singly, as blends or mixtures of essential oils, or in combination with other fragrancing constituents which may be synthetically produced or naturally derived, but need not be derived from or contain essential oils per se. Frequently, due to their potency, essential oils are often supplied dispersed in a liquid carrier such as in one or more organic solvents in which the essential oils are dissolved or dispersed.

By way of non-limiting example, exemplary useful essential oils providing an aromatherapy benefit which may find use in the topical compositions of the invention include: Abies Sibirica oil, Amyris Balsamifera oil, Anise oil, Balm Mint oil, Basil oil, Bay oil, Bee Balm oil, Bergamot oil, Birch oil, Bitter Orange oil, Cabbage Rose oil, Calendula Officinalis oil, California Nutmeg oil, Camellia Sinensis oil, Capsicum Frutescers oleoresin, Caraway Oil, Cardamon Oil, Cedarwood Oil, Chamaecyparis Obtusa Oil, Chamomile Oil, Cinnamon Oil, Citronella Oil, Clary Oil, Clove Oil, Cloveleaf Oil, Coriander Oil, Coriander Seed Oil, Cyperus Esculentus Oil, Cypress Oil, Eucalyptus Citriodora Oil, Eucalyptus Globulus Oil, Fennel Oil, Gardenia Florida Oil, Geranium Maculatum Oil, Ginger Oil, Grapefruit Oil, Hops Oil, Hypericum Perforatum Oil, Hyptis Suaveolens Oil, Indigo Bush Oil, Jasmine Oil, Juniperus Communis Oil, Juniperus Virginiana Oil, Labdanum Oil, Laurel Oil, Lavandin Oil, Lavender Oil, Lemon Oil, Lemongrass Oil, Leptospermum Scoparium Oil, Lime Oil, Linden Oil, Litsea Cubeba Oil, Lovage Oil, Mandarin Orange Oil, Massoy Bark Oil, Matricaria Oil, Moroccan Chamomile Oil, Musk Rose Oil, Myrrh Oil, Myrtle Oil, Norway Spruce Oil, Nutmeg Oil, Olax Dissitiflora Oil, Olibanum, Opoponax Oil, Orange Flower Oil, Orange Oil, Palmarosa Oil, Parsley Seed Oil, Passionflower Oil, Patchouli Oil, Pelargonium Graveolens Oil, Peppermint Oil, Pine Oil, Pine Tar Oil, Pine Kernel Oil, Pine Oil, Pine Cone Oil, Rosemary Oil, Rose Oil, Rosewood Oil, Rue Oil, Sage Oil, Sambucus Nigra Oil, Sandalwood Oil, Sandarac Gum, Sassafras Officinale Oil, Sisymbrium Ino Oil, Spearmint Oil, Sweet Marjoram Oil, Sweet Violet Oil, Tar Oil, Tea tree oil, Thyme Oil, Vetiveria Zizanoides Oil, Wild Mint Oil, Ximenia Americana Oil, Yarrow Oil, Ylang Yang Oil, or any combinations thereof.

Essential oils providing an aromatherapy benefit which may be used in the topical compositions of the present invention include one or more selected from chamomile oil, lavendin oil, lavender oil, grapefruit oil, lemon oil, line oil, mandarin orange oil, orange flower oil and orange oil. Chamomile oil may be used to promote both a fresh, clean and attractive scent and possibly provide a stress-relaxing benefit to the user of the topical composition. Lavender oil, and lavendin, may be used to promote both a fresh and attractive scent and possibly also provide a stress-relaxing benefit to the user of the topical composition. One or more of grapefruit oil, lemon oil, line oil, mandarin orange oil, orange flower oil and orange oil provide a clean citrus scent and may possibly impart a perceived therapeutic benefit as well when used.

These one or more essential oils providing an aromatherapy benefit may be used in the compositions of the invention in an amount of about 0.00001 wt. % to about 1 wt. %, based on the total weight of the composition. Preferably, the one or more essential oils providing an aromatherapy benefit are present in an amount about 0.00005 wt. % to about 0.75 wt. %, and more preferably about 0.0001 wt. % to about 0.5 wt. % of the total weight of the composition. It is to be understood that these one or more essential oils providing an aromatherapy benefit may be used with or without the optional fragrancing constituent recited previously and may be used wholly or partially in place of said fragrancing constituent.

The inventive compositions may include one or more colorants, e.g., dyes or pigments which are known to the art to be useful in cosmetic or topical compositions to impart a desired color or tint to the inventive compositions. Any colorant which is compatible with the other constituents forming the topical compositions may be used and such may be present in any amount effective to achieve the desired visual effect. Exemplary colorants include pigments, inter alia, inorganic red pigments, such as iron oxide, iron hydroxide and iron titanate; inorganic brown pigments, such as .gamma.-iron oxide; inorganic yellow pigments, such as iron oxide yellow and loess; inorganic black pigments, such as iron oxide black and carbon black; inorganic violet pigments, such as manganese violet and cobalt violet; inorganic green pigments, such as chromium hydroxide, chromium oxide, cobalt oxide and cobalt titanate; inorganic blue pigments, such as Prussian blue and ultramarine blue; lakes of tar pigments; lakes of natural dyes; and synthetic resin powder complexes of the inorganic pigments as recited above. Advantageously one or more colorants may be added in amounts of about 0.001% wt. to about 0.1% by weight, based on the total weight of the composition of which the colorant(s) forms a part.

The compositions of the invention may include one or more preservatives. It is preferred that the compositions of the invention comprise at least one preservative. Preservatives can be added to inhibit the growth of bacteria, fungi and/or yeasts and many suitable preservatives are known in the art. In the compositions of the invention it is especially preferred that a preservative which is effective against fungi and/or moulds is included therein. Exemplary useful preservatives include benzoates, such as sodium benzoate. Parabens may also be used but are less preferred than benzoates. The compositions of the invention may also include a material which is able to boost the effect of the preservative. These materials are herein termed “preservative booster”. Such preservative boosters predominantly exhibit other characteristics, but, do exhibit some antimicrobial effect although not necessarily sufficient effect to be used alone to provide a preservative effect in the composition, or, an antimicrobial effect for the compositions. Alcohols, such as glycols, may exhibit this preservative booster effect. Alkyl glycols, especially C₂-C₁₀ glycols in particular may exhibit this preservative booster effect. One such example is caprylyl glycol which is a preferred ingredient of the compositions of the invention. Caprylyl glycol's main function is to provide emolliency properties, but, as it has some antimicrobial effect it also assists the preservative and thus provides a boost in preservative power to the compositions. A suitable caprylyl glycol commercially available product is Microcare® CLG (ex Thor). Other suitable preservative boosters include benzyl alcohol. When present, the preservative is included in any amount found to be effective in retarding or inhibiting the grown of undesired microorganisms in the compositions of the invention, particularly during storage for several months at room temperature. The preservative is advantageously present in amounts of up to about 1.5% wt., preferably are present in amounts of from about 0.001% wt. to about 1.0% wt., and most preferably is present in an amount of from about 0.01% wt. to 0.75% wt, such as from about 0.01% wt. to 0.5% wt, especially as from about 0.1% wt to 0.3% wt based on the total weight of the composition of which it forms a part. The preservative booster is advantageously present in amounts of up to about 1% wt., preferably are present in amounts of from about 0.001% wt. to about 0.5% wt., and most preferably is present in an amount of from about 0.01% wt. to 0.25% wt, such as from about 0.01% wt. to 0.2% wt based on the total weight of the composition of which it forms a part.

A foam booster (which improves the foaming characteristics of the surfactant(s) present, especially anionic surfactant) may also be included. Preferred foam boosters are based on one or more alkanolamides which provide composition thickening, foam enhancement, and foam stability and in preferred embodiments of the invention are necessarily present. Exemplary alkanolamides which provide such a foam boosting function include but are not limited to: cocamide MEA, cocamide DEA, soyamide DEA, lauramide DEA, oleamide MIPA, stearamide MEA, myristamide MEA, lauramide MEA, capramide DEA, ricinoleamide DEA, myristamide DEA, stearamide DEA, oleylamide DEA, tallowamide DEA, lauramide MIPA, tallowamide MEA, isostearamide DEA, isostearamide MEA, and mixtures thereof. When present, the one or more alkanolamides are present in amounts of up to about 10% wt., but, are preferably included in amounts of from about 0.1-10% wt, such as 0.2-5% wt, for example 0.5-2.5% wt based on the total weight of the topical composition of which they form a part.

The compositions of the invention may optionally comprise one further antimicrobially active compounds or materials which are effective against gram-negative and/or gram-positive bacteria, and which are compatible with the other constituents present in the composition. Exemplary useful compounds and materials which may be used as the further antimicrobially active compound or material include one or more of one or more antimicrobial agents including: pyrithiones, (especially zinc pyrithione which is also known as ZPT), dimethyldimethylol hydantoin (Glydant®), methylchloroisothiazolinone/methylisothiazolinone (Kathon CG®), sodium sulfite, sodium bisulfite, imidazolidinyl urea (Germalil 1154), diazolidinyl urea (Germaill II®), benzyl alcohol, 2-bromo-2-nitropropane-1,3-diol (Bronopol®), formalin (formaldehyde), iodopropenyl butylcarbamate (Polyphase P100®)), chloroacetamide, methanamine, methyldibromonitrile glutaronitrile (1,2-Dibromo-2,4-dicyanobutane or Tektamer®), glutaraldehyde, 5-bromo-5-nitro-1,3-dioxane (Bronidox®), phenethyl alcohol, o-phenylphenol/sodium o-phenylphenol, sodium hydroxymethylglycinate (Suttocide A®), polymethoxy bicyclic oxazolidine (Nuosept C®), dimethoxane, thimersal dichlorobenzyl alcohol, captan, chlorphenenesin, dichlorophene, chlorbutanol, glyceryl laurate, halogenated diphenyl ethers for example 2,4,4′-trichloro-2′-hydroxy-diphenyl ether (Triclosan® or TCS), phenolic compounds like phenol and aromatic halophenols, 2-hydroxydiphenylmethane, resorcinol and its derivatives, bisphenolic compounds, benzoic esters (parabens) and halogenated carbanilides.

The one or more one further antimicrobially active compounds or materials may be present in amounts of from about from 0.001-3% wt., preferably in amounts from 0.1-2% wt., but are most desirably present from about 0.1-0.5% wt. based on the total weight of the composition of which they form a part.

The topical compositions may comprise one or more humectants, including polyhydric alcohols including polyalkylene glycols as well as alkylene polyols and their derivatives, inter alia, including propylene glycol, dipropylene glycol, polypropylene glycol, polyethylene glycol and derivatives thereof, sorbitol, hydroxypropyl sorbitol, erythritol, threitol, pentaerythritol, xylitol, glucitol, mannitol, hexylene glycol, butylene glycol (e.g., 1,3-butylene glycol), hexane triol (e.g., 1,2,6-hexanetriol), glycerine, ethoxylated glycerine and propoxylated glycerine. Further useful humectants include sodium 2-pyrrolidone-5-carboxylate, guanidine; glycolic acid and glycolate salts (e.g. ammonium and quaternary alkyl ammonium); lactic acid and lactate salts (e.g. ammonium and quaternary alkyl ammonium); aloe vera in any of its variety of forms (e.g., aloe vera gel); hyaluronic acid and derivatives thereof (e.g., salt derivatives such as sodium hyaluronate); lactamide monoethanolamine; acetamide monoethanolamine; urea; and, panthenol. The humectants may be used singly, or, two or more humectants may be included in topical compositions of the invention. Of the humectants, aloe vera in one or more of its forms is preferred as being a naturally derived product. When present, in accordance with certain of the preferred embodiments, one or more humectants may be included in effective amounts, advantageously from 0.01-2.5% wt., preferably from 0.01-2% wt. based on the total weight of the composition of which it forms a part.

The compositions of the invention may include one or more cationic Polyquaternium-type polymer if it is desired to impart moisturizing or conditioning properties to the compositions of the invention. Such materials may also have mild antimicrobial effects dependent upon the Polyquaternium polymer used. Such materials, are, per se, well known to the art of topical compositions.

The one or more cationic Polyquaternium-type polymers may be present in amounts of from about from 0.001-2.5% wt., preferably in amounts from 0.01-2% wt., but are most desirably present in weight percentages from about 0.05-1% wt. based on the total weight of the composition of which they form a part.

One optional constituent which may be included in the inventive compositions is a latex which may be used as an opacifier to provide opacification of the composition. Such are materials which are typically emulsions, dispersions or suspensions of a water insoluble polymer or copolymer in a carrier. Any suitable commercial opacifier may be used if an opaque composition is desired, such as those available under the trademark ACUSOL (ex. Rohm & Haas Inc.). When present in a composition according to the present invention, the opacifier may be present in amounts of up to about 5% wt., preferably are present in amounts of from about 0.001% wt. to about 3% wt., preferably are present in amount from about 0.1% wt. to about 1.2% wt, and most preferably are present in amounts of from about 0.1% wt. to about 1% wt., based on the total weight of the topical composition of which it forms a part.

The topical compositions may include one or more antioxidant constituents. Examples of antioxidants include but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, glutathione, and ascorbic acid and ascorbic acid derivatives (e.g., ascorbyl palmitate and ascorbyl polypeptide). Oil-soluble antioxidants suitable for use in the compositions of this invention include, but are not limited to, butylated hydroxytoluene, retinoids, tocopherols e.g., tocopherol acetate, tocotrienols, and ubiquinone. Natural extracts containing antioxidants suitable for use in the topical compositions of this invention, include but not limited to, extracts containing flavonoids and isoflavonoids and their derivatives, extracts containing resveratrol and the like. Examples of such natural extracts include grape seed, green tea, pine bark, propolis, and the like. When present the total amount of such antioxidants are usually not in excess of 5% wt, preferably are present in amounts of from 0.0001-4% wt. based on the total weight of the topical composition of which it forms a part.

The compositions may comprise a thickener, if required, to achieve the desired viscosity for the compositions. Any suitable thickener may be included in conventional amounts. Typically a polysaccharide based thickener may be included e.g., cellulose, alkyl celluloses, alkoxy celluloses, hydroxy alkyl celluloses, alkyl hydroxy alkyl celluloses, carboxy alkyl celluloses, carboxy alkyl hydroxy alkyl celluloses, and derivatives thereof including methyl cellulose ethyl cellulose, hydroxymethyl cellulose, hydroxy ethyl cellulose, hydroxy propyl cellulose, carboxy methyl cellulose, carboxy methyl hydroxyethyl cellulose, hydroxypropyl cellulose, hydroxy propyl methyl cellulose, ethylhydroxymethyl cellulose and ethyl hydroxy ethyl cellulose. Also suitable are naturally occurring polysaccharide polymers such as xanthan gum, guar gum, locust bean gum, tragacanth gum, or derivatives thereof, polycarboxylate polymers, polyacrylamides, clays, and mixtures thereof.

The polysaccharide-based thickener constituent, particularly the cellulose based thickener constituent, may be present in any effective amount, and are preferably present in amounts of from 0.01% wt. to 7.5% wt. based on the total weight of the composition.

The topical compositions may include one or more light stabilizers as well as UV absorbers. Such materials are known to be useful in cosmetic or topical compositions and impart a degree of stability to the compositions which may comprise one or more components which may be deleteriously affected when exposed to certain sources of light, e.g., sunlight, fluorescent light sources. Other such materials are known to stabilize or improve the effect of colorants which may be present in the compositions. Any cosmetically acceptable material or compound which provides protection for one or more of the constituents in the inventive compositions from photolytic degradation or photo-oxidative degradation may be used. When present, the one or more light stabilizers as well as UV absorbers may be included in any effective amount; advantageously such materials are present in amounts of from 0.0001-1% wt., preferably from 0.001-0.25% wt. based on the total weight of the composition of which it forms a part.

The compositions of the invention may include one or more chelating agents. Exemplary useful chelating agents include those known to the art, including by way of non-limiting example; aminopolycarboxylic acids and salts thereof wherein the amino nitrogen has attached thereto two or more substituent groups. Preferred chelating agents include acids and salts, especially the sodium and potassium salts of ethylenediaminetetraacetic acid, diethylenetriaminepentaacetic acid, N-hydroxyethylethylenediaminetriacetic acid, and of which the sodium salts of ethylenediaminetetraacetic acid may be particularly advantageously used. Such chelating agents may be omitted, or they may be included in generally minor amounts such as from 0.001-0.5% wt. based on the weight of the chelating agents and/or salt forms thereof. Desirably, such chelating agents are included in the present inventive composition in amounts from 0.01-0.5% wt., but are most desirably present in reduced weight percentages from about 0.01-0.2% wt.

Other extracts may also be included in the compositions of the invention, such as plant or flower extracts, oils or essences, to provide desirable benefits such as additional antimicrobial properties (for example tea tree oil as mentioned hereinabove), moisturizing properties, soothing properties, conditioning properties and/or fragrance. Suitable examples include plant extracts such as cucumber, mint and other herbs, and plant extracts such as honeysuckle, mugwort, marigold and calendula. If present such extracts may be included in conventional amounts and are usually not in excess of 2% wt, preferably are present in amounts of from 0.0001-1% wt, such as 0.01 to 0.5% wt, based on the total weight of the topical composition of which it forms a part.

According to some embodiments of the invention, the compositions may comprise alcohol, such as ethanol or propanol (including propan-1-ol and propan-2-ol). If present, the alcohol is preferably present in an amount of 0.1 wt % to 20 wt %, more preferably 0.5% wt % to 10% wt %, most preferably 1 wt % to 5 wt %. In other embodiments of the invention it is preferred that the compositions comprise less than 5 wt % alcohol, preferably less than 2.5 wt % and most preferably that they are substantially free of alcohol and especially that they are free of alcohol.

### Format of the Compositions of the Invention

The topical compositions of the invention are aqueous liquids. Preferably the aqueous liquids are foaming topical compositions meaning they produce foam during use.

Typically, the compositions of the inventions are clear or transparent liquid compositions, such as a clear or transparent liquid hand sanitizing composition. If it desired to make the composition opaque, an opacifier may be added. Typically, the compositions of the invention will be colorless or almost colorless. If a colored composition is required, suitable colorants may be added.

The liquid compositions of the invention are intended to provide antimicrobial benefits to the skin and/or hair and thus will be formulated in a physical form suitable for this purpose. Typically, the compositions of the invention are not structured liquids.

The liquid compositions of the invention are typically clear, or almost clear in appearance. They can however, if desired, be formulated as a lotion, a cream, an emulsion (including a microemulsion), mousse or a gel, which may be transparent, translucent or opaque as required.

The compositions of the invention may exhibit a viscosity around that of water (8.9×10⁴ Pa·s at about 25° C.) and for some applications this is preferred. It is also possible to formulate the compositions to have a higher viscosity (e.g. by the inclusion of thickeners) as desired.

The compositions of the invention may be provided in the form of a concentrated composition which is diluted prior to use to form a composition according to the invention. This provides environmental benefits as it avoids the unnecessary transportation of dilute compositions as the compositions are diluted after transportation but prior to use.

Generally, the compositions are formulated as liquid sanitizer products, especially hand and/or body sanitizer products and these are preferred according to the invention. The compositions of the invention may be formulated such that they do not require rinsing off the area to which they have been applied. However, according to one embodiment of the invention, a rinse-off composition is provided and compositions according to the invention can be formulated and used accordingly. Another preferred application for the composition of the invention is as a topical skin wound treatment composition (sanitizer) which can be applied directly to a minor skin wound or graze, and the surrounding area, for example shortly after the wound has been received to provide an initial antimicrobial effect thus reducing the chances of infection. The wound could be on the skin anywhere on the body. Hair sanitizer products can also be provided according to the present invention. The compositions of the invention are not specifically intended as treatment agents for the eyes or other mucosal membranes.

As well as being formulated specifically as sanitizing products for the hair and/or skin, other forms and other uses of the present compositions, such as face, hand or body lotion or cream, cleansing cream, massage materials, liquid soap, as well as hair care products such as shampoo, or other hair or scalp treatments are expressly contemplated as being within the scope of the present invention.

The topical compositions of the invention are intended for application to the skin and/or hair to provide an antimicrobial benefit thereto. Thus, typically the compositions are provided in a dispenser, or held in, or on, a substrate (such as a non-woven material) for use in such a treatment.

The composition can be packaged in any suitable dispenser to suit its viscosity and intended use by the consumer. Suitable dispensers for the compositions of the invention include spray dispensers, pump dispensers, aerosol containers, non-deformable dispensers and squeezable dispensers (where pressure is applied to the body of the dispenser, typically manual pressure) to achieve the egress of the composition from the dispenser. Squeezable dispensers are especially preferred for use with the compositions of the invention as they are generally convenient for the user to transport and use and can be produced in a suitable size, e.g. which can be squeezed with one hand to dispense the contents of the dispenser directly onto an area of skin (such as the other hand). Suitable such dispensers are well known in the art. According to one embodiment, the compositions may be provided in a single use container which is ruptured prior to use, e.g. in a polymer shell which is ruptured or dissolved prior to use. The polymer shell is compatible with the composition of the invention.

Suitable substrates which the compositions of the invention may be held in, or on, include wipes which are intended for single use and which are impregnated (or otherwise carry) a composition. Such wipes may be produced from non-woven substrate materials which are well known in the art and include those based on viscose, cotton, cellulose or cellulose derived materials. These items are typically referred to as ‘disposable wipes’. The substrate material is typically soaked in, impregnated with or sprayed with the composition to be delivered by the use of the wipe.

Thus, a second embodiment of the present invention provides a dispenser, especially a closed dispenser, containing a composition according to the present invention.

Thus, a third embodiment of the invention provides a disposable wipe comprising a composition of the invention.

### Use of the Compositions of the Invention

It is to be further expressly understood that topical application of the topical composition disclosed herein may be applied to the skin on any part of the body, including the skin on the face, neck, chest, back, arms, axilla, hands, legs, and scalp. It may also be applied to hair if it is desired to impart an antimicrobial effect to hair. However, primarily, the topical compositions of the invention are intended for application to the skin on any part of the body.

Use of the topical compositions of the invention on the hands is especially preferred and provides a rapid, effective and convenient method of sanitizing the hands and according to one embodiment of the invention without the need for soap and water. Thus, the compositions of the invention provide a convenient method of effectively sanitizing any part of the body, especially the hands, in situations where there is no access to water and/or where a rapid method of hand sanitization is required. Hands, in particular, may require frequent sanitization and thus the present invention may be packaged in containers which can be carried by the user to be used as and when sanitization is required.

To use the compositions of the invention, the user may simply dispense the desired quantity of the compositions from the container in which they are held onto the part of the body to be sanitized (e.g. the hands) and rubs the compositions around the applicable body part. The foaming nature of compositions according to the invention allows the user to visually and/or by feel assess the coverage of the composition on that body part and move the composition around as necessary to provide for good coverage and hence effective sanitization.

The area of the body to which the composition of the invention is applied may be dry or may have been wetted with water prior to application of the topical composition. For non-rinse compositions the hands will generally be dry or substantially dry.

The compositions of the invention according to one embodiment of the invention do not need to be rinsed off, or, removed by drying. However, the user may do either, or both, of these actions if they so choose. According to another embodiment of the invention, compositions are provided which are intended to be rinsed off after use. These compositions would typically be used instead of traditional bar or liquid soaps and in applications where rinsing water was available. After use the treated area of skin or hair is permitted to air dry, or, it may be manually dried e.g. by the use of a fabric or paper.

Contact times between the compositions of the invention and the area of skin or hair to be sanitized of around 60 seconds are preferred to provide optimum contact time for the antimicrobial effect to be imparted. However, contact times of up to 45 seconds, seconds or even up to 20 or 10 seconds can also be used to provide effective sanitization of the area being treated.

Thus, in a fourth embodiment, the present invention provides the use of the topical compositions of the present invention to sanitize skin and/or hair by the topical application thereto. It is especially preferred that the compositions of the present invention are used to sanitize skin on the hands.

In a fifth embodiment, the present invention provides a topical method of sanitizing skin and/or hair by providing an antimicrobial effect by means of applying to the area to be sanitized a composition according to the invention in a suitable amount to provide a sanitizing effect and allowing contact between the composition and the skin and/or hair to be treated for sufficient time to provide the antimicrobial effect thereon. Preferably the composition is applied to skin, and most especially to skin on the hands.

### Antimicrobial Activity of the Compositions

The compositions of the present invention provide effective antimicrobial activity (germ killing properties) upon both gram-positive bacteria (such as Staphylococcus aureus (S. aureus) and gram-negative bacteria Escherichia coli (such as E. coli). The antimicrobial action of the compositions of the invention has been tested using the specified bacterial strains but other suitable strains could be employed instead of the stated strains to demonstrate the antimicrobial effectiveness of the compositions of the invention.

The inventors have observed that tricarboxylic acids, especially citric acid, are very effective upon gram-negative bacteria (such as E. Coli), but, slightly less effective against gram-positive bacteria such as S. aureus. The inventors have also observed that monocarboxylic acids, especially lactic acid, are effective against gram-positive bacteria (such as S. aureus) and help to provide a good level of antimicrobial activity for the composition against both gram-negative bacteria and gram-positive bacteria when used with a tricarboxylic acid. Therefore, it is especially preferred for the compositions of the present invention to comprise both at least one tricarboxylic acid and at least one monocarboxylic acid.

For the antimicrobial testing results of the compositions given in the Examples, while no established criteria are considered a standard for a “pass” or “fail” determination in tests to establish the antimicrobial activity of the compositions of the invention, it is considered by the inventors that any tested formulation which fails to provide a log₁₀ reduction of less than 3 is a “fail” score, while any formulation which provided a logic reduction of 3 or more is considered a “pass” score and therefore a superior performing formulation against the stated pathogens, in this case the stated gram-positive and gram-negative bacteria. In particular, the inventors found that formulations which achieved scores of 3.5 or more, especially 4 or more, most especially 4.5 or more were much preferred as they increasingly exhibited superior antimicrobial effects as the score increased. The most preferred compositions of the invention achieved a log₁₀ reduction score of 5 or more.

The compositions of the invention are also believed to be effective against spores and certain viruses, especially enveloped viruses, such as those which cause the common cold and influenza.

### Preparation of the Compositions

The compositions of the invention may be produced by any suitable method. One suitable method which may be used to produce the compositions is;
1. Add approximately 50% of the total amount of distilled water in the composition at ambient temperature into a suitable mixing tank and stir with a mechanical stirrer at a sufficient speed to produce a vortex.
2. Add the required amount of surfactant (e.g. sodium lauryl ether sulphate) to the mixing tank and mix well (e.g. at a speed sufficient to maintain the vortex, for about 10 minutes).
3. If using a material which aids the preservative system, such as caprylyl glycol, add the required amount (pre-heated to a temperature of approximately 35° C. to 40° C. if required to aid addition) to the mixing tank and mix for about a further 10 minutes or until the material is fully dispersed in the water/surfactant mixture.
4. Add approximately 40% of the total amount of distilled water in the composition to the mixing tank at ambient temperature into the mixing tank and continue mixing until the resultant composition is well mixed.
5. Add the required amount of a pre-prepared solution of carboxylic acid (e.g. a 50% solution of citric acid) to the mixing tank and continue mixing until the resultant composition is well mixed.
6. If using a second carboxylic acid, add the required amount of a pre-prepared solution of the second carboxylic acid (e.g. a 50% solution of lactic acid) to the mixing tank and continue mixing until the resultant composition is well mixed.
7. Add the required amount of fragrance to the mixing tank and continue mixing until the resultant composition is well mixed.
8. Add the required amount of preservative (e.g. sodium benzoate) to the mixing tank and continue mixing until the resultant composition is well mixed.
9. Measure the pH of the composition and adjust to a pH within the target range for the composition according to the invention using a suitable base (e.g. a 30% wt solution of sodium hydroxide), noting the amount used to achieve the desired pH. Continue mixing the composition until well mixed.
10. Add an amount of deionized water sufficient to make the composition up to 100 parts (water to 100%, or q.s.).

The following examples below illustrate exemplary formulations as well as preferred embodiments of the invention. It is to be understood that these examples are provided by way of illustration only and that further useful formulations falling within the scope of the present invention and the claims may be readily produced by one skilled in the art without deviating from the scope and spirit of the invention.

### EXAMPLES

Liquid aqueous compositions according to the invention are given in the following examples. The compositions are all intended for topical application to skin/hair. They find particular application as skin sanitizers and especially as hand sanitizers. These compositions were prepared following the method of preparation stated hereinabove.

In the following compositions, the constituents were used “as supplied” from their respective suppliers and may constitute less than 100% wt. “actives”, or, may have been supplied as constituting 100% wt. “active” of the named compound, as indicated in the following tables.

The percentages given in the tables are as % wt. based on the total weight of the compositions.

In each of the compositions deionized water was included in “quantum sufficient” (q.s.) to provide 100 parts by weight of the specific composition as stated in the tables.

For each composition in Table 1, the amount of 30% sodium hydroxide solution noted added is the amount added to bring the composition from its initial unadjusted pH to the pH stated in Table 1 as the “final pH”.

Comparative examples which are outside of the scope of the invention are numbered with the prefix “C” followed by a number.

The compositions above in the tables below 1 were evaluated for their antimicrobial efficacy against Staphylococcus aureus (ATCC 6538) and Escherichia coli (ATCC 10536).

The test method used to evaluate the antimicrobial effectiveness of the Examples was an antimicrobial suspension test based on British standard reference No: EN 1276: 2009 method entitled “Chemical Disinfectants and Antiseptics—Quantitative Suspension Test for the Evaluation of bactericidal activity of chemical disinfectants and antiseptics used in food, industrial, domestic and institutional areas—Test method and requirements (Phase 2, step 1)” with the following modifications made to the method;
eliminating the interfering substance indicated by the protocol as “dirty or clean conditions” and
increasing the test temperature from 20±1° C. to 37±1° C. (as 37° C. is closer to the average human body temperature than 20° C.).

The test protocol tests the efficacy of the compositions of the present invention and the comparative examples against gram-negative and gram-positive bacteria. The test organisms used were Staphylococcus aureus ATCC 6538 and Escherichia coli ATCC 10536.

The bacterial strains were cultured on tryptic soy agar (TSA) slant from frozen stock and incubated for 24 hours. Following incubation, 2^{nd} and 3^{rd} generation transfers were prepared and used to prepare test suspensions as described in the EN 1276:2009 test method. The cell suspensions were adjusted to produce approximately 1.5-5.0×10⁸ CFU mL⁻¹. The growth medium and temperature used were Tryptic soy Agar (TSA) and 35±1° C. Test solutions and test cultures are equilibrated to a test temperature of 37≅1° C. in a water bath.

Experimentally, a 1.0 mL of the test culture was exposed to 9.0 mL of the test product for a 1-minute contact time and neutralized in a verified neutralizer. After a 5-minute neutralization time, the neutralized sample was serially diluted, plated on TSA and incubated at 35±1° C. for 48 hours. The average Log₁₀ CFU/mL for the test suspension was calculated and used to compute the log reduction post-treatment. A three log₁₀ reduction of all tested bacterial strains in a 1-minute contact time was chosen to indicate that the tested formulation has the desired level of antimicrobial properties against tested organism(s).

The formula applied to calculate the log reductions was: Log Nc−Log Nd where:

Nc=Number of cfu/ml of the test suspension count and Nd=Number of cfu/ml of the sample count after treatment.

TABLE 1
Effect of pH on antimicrobial activity of the compositions.

| | Ex. 1 | Ex. 2 | Ex. 3 | Ex. 4 | Ex. 5 | Ex. 6 | C1 | C2 | C3 |
|---|---|---|---|---|---|---|---|---|---|
| | % wt | % wt | % wt | % wt | % wt | % wt | % wt | % wt | % wt |
| Citric acid 50% | 3.8 | 3.8 | 3.8 | 3.0 | 4.8 | 6.0 | 3.8 | 3.0 | 3.8 |
| solution*¹ | | | | | | | | | |
| Lactic acid 50% | 1.0 | 1.0 | — | 1.0 | — | — | 1.0 | 1.0 | — |
| solution*¹ | | | | | | | | | |
| Sodium lauryl | 0.2 | 1.4 | 0.2 | 0.2 | 0.2 | 0.2 | 0.2 | 0.2 | 0.2 |
| ether sulfate | | | | | | | | | |
| (SLES) *² | | | | | | | | | |
| Sodium lauryl | — | — | — | — | — | — | — | — | — |
| sulfate (SLS) *² | | | | | | | | | |
| Sodium | — | — | 1.52 | 1.97 | 2.66 | 3.64 | 3.34 | 2.71 | 2.68 |
| hydroxide | | | | | | | | | |
| (30% w/w) | | | | | | | | | |
| solution*3 | | | | | | | | | |
| Sodium | 0.2 | 0.2 | 0.2 | 0.1 | 0.2 | 0.2 | 0.2 | 0.2 | 0.2 |
| benzoate | | | | | | | | | |
| Microcare ® | 0.1 | 0.1 | 0.1 | 0.1 | — | — | — | 0.1 | 0.1 |
| CLG | | | | | | | | | |
| Microcare ® | — | — | — | — | 0.2 | 0.2 | — | — | — |
| SBB | | | | | | | | | |
| D.I. water | q.s. | q.s. | q.s. | q.s. | q.s. | q.s. | qs. | q.s. | q.s. |
| Test results | | | | | | | | | |
| pH (final) | 3.95 | 4.02 | 4.07 | 4.2 | 4.49 | 4.59 | 4.93 | 4.95 | 5.10 |
| Log₁₀ reduction | >5.83 | 4.51 | >5.52 | >5.48 | 5.25 | 4.08 | 2.53 | 2.29 | <1.62 |
| for S. aureus. | | | | | | | | | |
| Log₁₀ reduction | >5.38 | >5.2 | >5.38 | >5.53 | >5.42 | >5.52 | 2.7 | 1.41 | <0.90 |
| for E. coli. | | | | | | | | | |

*¹As the Citric acid and Lactic acids were added as a 50% solution the active level is 50% of amount stated in the examples.
*² As the SLES and SLS were added as a 70% solution the active level is 70% of amount stated in the examples.
*3As the sodium hydroxide was added as a 30% solution the active level is 30% of amount stated in the examples.

TABLE 2
Effect of surfactant on antimicrobial activity of the compositions.

| | Ex. 2 | Ex. 7 | Ex. 8 | Ex. 9 | Ex. 10 | C4 |
|---|---|---|---|---|---|---|
| | % wt | % wt | % wt | % wt | % wt | % wt |
| Citric acid 50% solution*¹ | 3.8 | 4.0 | 4.0 | 4.0 | 4.0 | 4.0 |
| Lactic acid 50% solution*¹ | 1.0 | — | 1.0 | 1.0 | 1.0 | 1.0 |
| Sodium lauryl ether sulfate | 1.4 | 0.2 | 0.2 | — | — | — |
| (SLES) *² | | | | | | |
| Sodium lauryl sulfate | — | — | — | 0.2 | 0.1 | — |
| (SLS) *2 | | | | | | |
| Sodium hydroxide | — | 1.79 | 2.12 | 0.16 | 0.16 | 0.16 |
| (30% w/w) | | | | | | |
| solution*3 | | | | | | |
| Sodium benzoate | 0.2 | 0.1 | 0.2 | — | 0.2 | 0.2 |
| Microcare ® CLG | 0.1 | — | — | — | — | — |
| Microcare ® SBB | — | — | 0.1 | — | 0.1 | 0.1 |
| D.I. water | q.s. | q.s. | q.s. | q.s. | q.s. | q.s. |
| Test results | | | | | | |
| pH (final) | 4.02 | 4.15 | 3.97 | 3.97 | 4.04 | 4.15 |
| Log₁₀ reduction | 4.51 | 5.37 | >5.48 | >5.2 | >5.25 | <1.94 |
| for S. aureus. | | | | | | |
| Log₁₀ reduction | >5.2 | >5.53 | >5.53 | >5.47 | >5.25 | <1.94 |
| for E. coli. | | | | | | |

The identity of the specific constituents used to produce the foregoing Examples is recited in Table 3.

| Constituent | Function | Details |
|---|---|---|
| D.I. water | Solvent | Distilled water |
| Citric acid 50% | Antimicrobial | A 50% wt solution of citric |
| solution | agent | acid (98-100% wt, actives) |
| | | provided as anhydrous citric |
| | | acid (ex COFOC (Shashi)) and |
| | | made into a 50% solution. |
| Lactic acid 50% | Antimicrobial | A 50% wt solution of lactic |
| solution | agent | acid (Purse ® FCC 50 ex |
| | | Purac). |
| Sodium lauryl | Surfactant | Anionic surfactant. 70% wt. |
| ether sulfate | | active solution. (ex. Zhisheng |
| (SLES) | | (HuiZhou)) |
| Sodium lauryl | Surfactant | Anionic surfactant. 70% wt. |
| sulfete (SLS) | | active solution. |
| Sodium | pH adjuster | A 30% solution of sodium |
| hydroxide | | hydroxide (98-100% wt. active) |
| (30% w/w) | | provided as anhydrous |
| solution | | sodium hydroxide. |
| Sodium | Preservative | Used as the powder. Available |
| benzoate | against yeasts | commercially from a variety |
| | and fungi | of suppliers. |
| Earth Rose | fragrance | proprietary composition of |
| fragrance | | its supplier |
| Citromax ® | fragrance | proprietary composition of |
| fragrance | | its supplier |
| 446959 | | |
| Microcare ® | Emollient and aids | caprylyl glycol (ex Thor) |
| CLG | preservative effect | |
| Microcare ® | Preservative | A liquid blend of benzyl |
| SBB | | alcohol and two organic |
| | | acids (ex Thor) |

### Results

The results in Table 1 (effect of pH) demonstrate that the compositions of the present invention show excellent antimicrobial activity on both S. aureus and E. coli in the pH range 3.95 to 4.59 for examples 1-6. All these samples are considered by the inventors to pass the test for acceptable antimicrobial activity.

The comparative examples C1-C3, which have a pH in the range 4.93 to 5.10, all exhibit poor antimicrobial activity on both S. aureus and E. coli and are considered by the inventors to fail the test for acceptable antimicrobial activity.

It can also be appreciated from the compositions in Table 1 that a pH below 3.95 would also be acceptable according to the present invention. There is, of course a balance to be had for the compositions of the invention between antimicrobial activity and skin tolerance. Hence very low pH's (especially below pH 3) are avoided as they are more likely to adversely affect the skin/hair.

It can also be seen from the results in Table 1 that compositions which comprise only citric acid as the carboxylic acid (examples 5 and 6), are especially effective at pHs towards the upper end of the pH range of the present invention. When the compositions comprise both citric acid and lactic acid (examples 1, 2 and 4) excellent results were obtained for antimicrobial activity at pHs towards the lower end of the pH range of the present invention.

Additionally, it can be seen from the examples that the absence or presence of the optional ingredients Microcare® CLG and Microcare® SBB has no significant bearing on the antimicrobial activity of the compositions.

The results in Table 2 (effect of surfactant) demonstrate that in the absence of the surfactant ineffective antimicrobial action is exhibited by the compositions (see e.g. comparative example C4 compared to Example 10). Examples 2 and 7 to 8, which are all according to the present invention, all show excellent antimicrobial activity. The concentrations of surfactant range (in active %) from 0.07% wt. to 0.98% wt based on the total weight of the composition.

The inventors have also noticed that compositions comprising a greater amount of citric acid, than lactic acid, when both are present in the compositions are especially advantageous for antimicrobial effects and skin tolerance. In the above examples the ratio of citric acid:lactic acid in the range of from 4:1 to 3:1 show especially good antimicrobial effects.

While the invention is susceptible of various modifications and alternative forms, it is to be understood that specific embodiments thereof have been shown by way of example which are not intended to limit the invention to the particular forms disclosed; on the contrary the intention is to cover all modifications, equivalents and alternatives falling within the scope and spirit of the invention as expressed in the appended claims.

1. An aqueous liquid composition comprising:
0.001% wt. to 1% wt. surfactant; and
at least one carboxylic acid or a salt thereof,
wherein the aqueous liquid composition is at a pH of about 4.7 or less, and
wherein the aqueous liquid composition provides a topical antimicrobial benefit.

2. The aqueous liquid composition according to claim 1, wherein the aqueous liquid composition is a foaming composition.

3. The aqueous liquid composition according to claim 1, wherein the composition comprises 85% wt to 99% wt water, based on the total weight of the composition.

4. The aqueous liquid composition according to claim 1, wherein the composition comprises 0.05% wt to 0.98% wt. surfactant based on the total weight of the composition.

5. The aqueous liquid composition according to claim 1, wherein the surfactant comprises an anionic surfactant.

6. The aqueous liquid composition according to claim 5, wherein the anionic surfactant comprises alkyl sulfates or salts thereof.

7. The aqueous liquid composition according to claim 6, wherein the alkyl sulfates or salts thereof are selected from the group consisting of C8-C18 alkyl sulfates and their salts, and C8-C18 alkyl ether sulfates and their salts.

8. The aqueous liquid composition according to claim 7, wherein the C8-C18 alkyl sulfate and its salts is lauryl sulfate and its salts, and the C8-C18 alkyl ether sulfates and their salts is lauryl ether sulfate and its salts.

9. The aqueous liquid composition according to claim 8, wherein the lauryl sulfate and its salts is sodium lauryl sulfate and the lauryl ether sulfate and its salts is sodium lauryl ether sulfate.

10. The aqueous liquid composition according to claim 1, wherein the composition comprises a nonionic surfactant and/or a cationic surfactant and/or an amphoteric surfactant.

11. The aqueous liquid composition according to claim 1, wherein the composition comprises less than about 5% wt of the carboxylic acid or salt thereof based on the total weight of the composition.

12. The aqueous liquid composition according to claim 11, wherein the composition comprises from 0.1% wt to 4.5% wt of the carboxylic acid or salt thereof.

13. The aqueous liquid composition according to claim 1, wherein the composition comprises a monocarboxylic acid or salt thereof.

14. The aqueous liquid composition according to claim 13, wherein the monocarboxylic acid or a salt thereof is present, in an amount of from 0.1% wt. to 4.5% wt based on the total weight of the composition.

15. The aqueous liquid composition according to claim 1, wherein the composition comprises a tricarboxylic acid or salt thereof.

16. The aqueous liquid composition according to claim 15, wherein the tricarboxylic acid or a salt thereof is present in an amount of from 0.1% wt. to 4.5% wt based on the total weight of the composition.

17. The aqueous liquid composition according to claim 1, wherein the composition comprises a tricarboxylic acid and a monocarboxylic acid.

18. The aqueous liquid composition according to claim 17, wherein the composition comprises the tricarboxylic acid and the monocarboxylic acid in a weight ratio of from 10:1 to 1:1.

19. The aqueous liquid composition according to claim 17, wherein the composition comprises citric acid and lactic acid.

20. The aqueous liquid composition according to claim 1, wherein the composition is at a pH of about 4.6 or less.

21. The aqueous liquid composition according to claim 20, wherein the composition is at a pH of from about 3 to about 4.6.

22. The aqueous liquid composition according to claim 21, wherein the composition is at a pH of from 3.5 to about 4.5.

23. The aqueous liquid composition according to claim 1, wherein the composition further comprises 0.1 wt % to 20 wt % alcohol.

24. The aqueous liquid composition according to claim 1, wherein the composition is substantially free of alcohol.

25. A dispenser containing the aqueous liquid composition according to claim 1.

26. A disposable wipe comprising the aqueous liquid composition according to claim 1.

27. A method of using the aqueous liquid composition according to claim 1 to provide an antimicrobial benefit to sanitize skin and/or hair comprising topically applying the aqueous liquid composition to the skin and/or hair.

28. A method for providing an antimicrobial benefit to skin and/or hair desired to be sanitized, comprising the steps of:
topically of applying the aqueous liquid composition according claim 1, to skin and/or hair to be sanitized in a suitable amount to provide a sanitizing effect, and
allowing contact between the aqueous liquid composition and the skin and/or hair for sufficient time to provide the antimicrobial effect thereon.
